(19) United States
(12) Patent Application Publication
(10) Pub. No.: US 2017/0335286 A1
(43) Pub. Date: Nov. 23, 2017

(54) METHODS FOR MAINTAINING AND EXPANDING MESENCHYMAL STEM CELLS ON EXTRACELLULAR MATRIX COATED MICROCARRIERS

(71) Applicant: STEMBIOSYS, INC., San Antonio, TX (US)

(72) Inventors: Rogelio ZAMILPA, San Antonio, TX (US); Edward S. GRIFFEY, San Antonio, TX (US)

(21) Appl. No.: 15/521,970

(22) PCT Filed: Oct. 30, 2015
(86) PCT No.: PCT/US2015/058335

§ 371 Date: Apr. 26, 2017

Related U.S. Application Data
(60) Provisional application No. 62/072,771, filed on Oct. 30, 2014.

(51) Int. Cl. C12N 5/0775

(52) U.S. Cl. CPC C12N 5/0662; C12N 5/0663; C12N 2537/10; C12N 2531/00; C12N 2513/00; C12N 2539/00; C12N 2533/90

(57) ABSTRACT

Disclosed are methods for coating microcarriers with a marrow stromal cell derived extracellular matrix, and maintaining and expanding mammalian mesenchymal stem cells on the marrow stromal cell derived extracellular matrix coated microcarriers in culture.

### CROSS REFERENCE TO RELATED APPLICATIONS

This application claims the benefit of U.S. Provisional Patent Application No. 62/072,771, filed Oct. 30, 2014, which is incorporated by reference herein in its entirety.

### BACKGROUND OF THE INVENTION

### A. Field of the Invention

The invention generally relates to methods of maintaining and expanding mammalian mesenchymal stem cells in culture in an undifferentiated state. In particular, the methods utilize bone marrow stromal cell derived extracellular matrix coated microcarriers as an in-vitro microenvironment for the maintenance and expansion of mesenchymal stem cells in an undifferentiated state.

### B. Description of Relevant Art

Mesenchymal stem cells (MSCs) are multipotent cells that can produce daughter stem cells and can also differentiate into a variety of cell types including, but not limited to osteoblasts, stromal cells that support hematopoiesis and osteoclastogenesis, chondrocytes, myocytes, adipocytes, neuronal cells, and B-pancreatic islet cells. MSCs can be isolated from small tissue samples and expanded in-vitro under cell culture conditions. Mammalian MSCs can be obtained from bone marrow, embryonic yolk sac, placenta, umbilical cord tissues, umbilical cord blood, periosteum, trabecular bone, adipose tissue, synovium, skeletal muscle, deciduous teeth, fetal pancreas, lung, liver, amniotic fluid, and fetal and adolescent skin and blood. Use of MSCs in therapeutic applications is desirable due to their capacity for self-renewal and multi-lineage differentiation. However, MSCs tend to lose their stem cell properties under conventional cell culture conditions, such as when cultured on tissue culture plastic. This situation has impaired the use of MSCs for therapeutic purposes

U.S. Pat. No. 8,084,023 and US patent publication 2013/0195814, both of which are herein incorporated by reference in their entirety, demonstrate that culture of mammalian MSCs on a three-dimensional (3D) extracellular matrix (ECM) made by marrow derived stromal cells promotes self-renewal of the MSCs and helps maintain the MSCs in an undifferentiated state. This particular ECM comprises collagen types I and III, syndecan-1, perlecan, fibronectin, laminin, biglycan, and decorin as identified by immunohistochemical staining. This ECM promotes self-renewal of MSCs, restrains their spontaneous differentiation toward the osteoblast lineage, and preserves their ability to differentiate into osteoblasts or adipocytes in response to BMP2 or rosiglitazone, respectively. The substrates used to grow these cells included containers, such as culture flasks or bioreactors, where the cells are cultivated on the inner planar surfaces of the containers. Notably, however, the methods disclosed in each of these references are limited by their respective yields of produced undifferentiated MSCs. In particular, the yields are not sufficient for large-scale commercial and therapeutic applications.

### SUMMARY OF THE INVENTION

The present invention provides a solution to the aforementioned limitations and deficiencies in the art relating to maintaining and expanding mammalian mesenchymal stem cells (MSCs) in culture in an undifferentiated state. The solution is premised on the use of microcarriers to serve as a substrate for a marrow stromal cell derived extracellular matrix (ECM). In particular, it was discovered in the context of the present invention that microcarriers coated with the marrow stromal cell derived ECM not only significantly increased the attachment surface area for adherent cells, but also allowed for faster and more efficient expansion of cultured MSCs than with previous methods while still maintaining the MSCs in an undifferentiated state. Without wishing to be bound by theory, it is believed that by closely reproducing the biochemical and ultrastructural microenvironment of the bone marrow stroma, the MSCs recognize a more natural environment (than plastic or other synthetic and biosynthetic materials commonly used for microcarriers) and proliferate more rapidly without differentiating to establish homeostasis of their natural environment. The features of the present invention allow for the production of yields of undifferentiated MSCs suitable for use in large-scale commercial and therapeutic applications.

In one aspect of the invention, there is disclosed a method of maintaining and expanding mammalian mesenchymal stem cells in culture in an undifferentiated state, the method comprising: producing a 3D extracellular matrix coating on the surface of microcarriers comprising: adding the microcarriers to a culture medium, adding mammalian marrow stromal cells to the culture medium, culturing the marrow stromal cells to produce the 3D extracellular matrix coating on the surface of the microcarriers, decellularizing the extracellular matrix coated microcarriers of the marrow stromal cells; and culturing the mammalian mesenchymal stem cells in the presence of the extracellular matrix coated microcarriers; wherein the extracellular matrix coating restrains differentiation of the mammalian mesenchymal stem cells.

Alternatively, there is disclosed a method of maintaining and expanding mammalian mesenchymal stem cells in culture in an undifferentiated state, the method comprising: obtaining marrow stromal cell derived 3D extracellular matrix coated microcarriers and culturing the mammalian mesenchymal stem cells in the presence of the extracellular matrix coated microcarriers, wherein the extracellular matrix coating restrains differentiation of the mammalian mesenchymal stem cells.

Still further, there is disclosed marrow stromal cell derived 3D extracellular matrix coated microcarriers. The microcarriers can be a plurality of microcarriers. The microcarriers can be free or substantially free of marrow stromal cells (e.g., by decellularizing the extracellular matrix coated microcarriers of the marrow stromal cells). The microcarriers can be combined with mammalian mesenchymal stem cells (e.g, MSCs attached to the microcarriers viand/or the 3D extracellular matrix). The microcarriers can be placed in a composition that promotes MSC expansion and maintenance (e.g., cell culture media).

In one embodiment, the extracellular matrix coating comprises type I collagen, type III collagen, fibronectin, decorin, biglycan, perlecan, and laminin. In another embodiment, the extracellular matrix coating comprises type I collagen, type III collagen, fibronectin, decorin, biglycan, perlecan, and laminin and further comprises at least one of syndecan-1, collagen type V, or collagen type VI. In another embodiment, the extracellular matrix coating comprises collagen alpha-1(XII), collagen alpha-3(VI), EMILIN-1, serpin H1, thrombospondin-1, tenascin precursor (TN) (Human), transforming growth factor-beta-induced protein, and vimentin. In another embodiment, the extracellular matrix coating comprises collagen alpha-1(XII), collagen alpha-3(VI), EMILIN-1, serpin H1, thrombospondin-1, tenascin precursor (TN) (Human), transforming growth factor-beta-induced protein, vimentin, type I collagen, type III collagen, fibronectin, decorin, biglycan, perlecan, and laminin. In another embodiment, the extracellular matrix coating comprises collagen alpha-1(XII), collagen alpha-3(VI), EMILIN-1, serpin H1, thrombospondin-1, tenascin precursor (TN) (Human), transforming growth factor-beta-induced protein, vimentin, type I collagen, type III collagen, fibronectin, decorin, biglycan, perlecan, and laminin, and further comprises at least one of syndecan-1, collagen type V, or collagen type VI.

In one embodiment, the marrow stromal cells are murine, rabbit, cat, dog, pig, or primate. In another embodiment, the marrow stromal cells are human.

In still another embodiment, the marrow stromal cells are isolated marrow mesenchymal stem cells.

In one embodiment, the mammalian mesenchymal stem cells are obtained from bone marrow. In another embodiment, the mammalian mesenchymal stem cells are obtained from umbilical cord blood.

In one embodiment, the microcarriers are spherical in shape. In another embodiment, the microcarriers are cylindrical in shape. A mixture of spherical and cylindrical shapes can also be used. In particular aspects, the cylindrical microcarriers are fibers. In another aspect, the cylindrical microcarriers are hollow fibers. In one embodiment, the microcarriers have a positive charge. In another embodiment, the microcarriers have a negative charge. In another particular embodiment, the microcarriers are spherical in shape, comprise a cross-linked dextran matrix and have a positive charge.

In one embodiment, the method further comprises culturing the marrow stromal cells or the mammalian mesenchymal stem cells, or both, under normoxic conditions.

In one aspect, the method further comprises culturing the marrow stromal cells or the mammalian mesenchymal stem cells, or both, in a container suitable for cell cultivation. In another embodiment, the container is a bioreactor.

Also disclosed in the context of the present invention are embodiments 1 to 50. Embodiment 1 is a method of maintaining and expanding mammalian mesenchymal stem cells in culture in an undifferentiated state, the method comprising producing a 3D extracellular matrix coating on the surface of microcarriers comprising adding the microcarriers to a culture medium; adding mammalian marrow stromal cells to the culture medium; culturing the marrow stromal cells to produce the extracellular matrix coating on the surface of the microcarriers; decellularizing the extracellular matrix coated microcarriers of the marrow stromal cells; and culturing the mammalian mesenchymal stem cells in the presence of the extracellular matrix coated microcarriers; wherein the extracellular matrix coating restrains differentiation of the mammalian mesenchymal stem cells. Embodiment 2 is the method of embodiment 1, wherein the extracellular matrix coating comprises collagen alpha-1 (XII), collagen alpha-3 (VI), EMILIN-1, serpin H1, thrombospondin-1, tenascin precursor (TN) (Human), transforming growth factor-beta-induced protein, and vimentin. Embodiment 3 is the method of embodiment 2, wherein the extracellular matrix coating further comprises type I collagen, type III collagen, fibronectin, decorin, biglycan, perlecan, and laminin. Embodiment 4 is the method of embodiment 3, wherein the extracellular matrix coating further comprises at least one of syndecan-1, collagen type V, or collagen type VI. Embodiment 5 is the method of any one of embodiments 1 to 4, wherein the marrow stromal cells are murine, rabbit, cat, dog, pig, or primate. Embodiment 6 is the method of any one of embodiments 1 to 4, wherein the marrow stromal cells are human. Embodiment 7 is the method of any one of embodiments 1 to 6, wherein the marrow stromal cells are isolated marrow mesenchymal stem cells. Embodiment 8 is the method of any one of embodiments 1 to 7, wherein the mammalian mesenchymal stem cells are obtained from bone marrow. Embodiment 9 is the method of any one of embodiments 1 to 7, wherein the mammalian mesenchymal stem cells are obtained from umbilical cord blood. Embodiment 10 is the method of the method of any one of embodiments 1 to 9, wherein the microcarriers are spherical in shape. Embodiment 11 is the method of the method of any one of embodiments 1 to 10, wherein the microcarriers have a positive charge. Embodiment 12 is the method of the method of any one of embodiments 1 to 10, wherein the microcarriers have a negative charge. Embodiment 13 is the method of embodiment 10, wherein the microcarriers comprise a cross-linked dextran matrix and have a positive charge. Embodiment 14 is the method of the method of any one of embodiments 1 to 9, wherein the microcarriers are cylindrical in shape. Embodiment 15 is the method of embodiment 14, wherein the microcarriers are fibers. Embodiment 16 is the method of embodiment 15, wherein the fibers are hollow fibers. Embodiment 17 is the method of the method of any one of embodiments 1 to 16, wherein the method further comprises culturing the marrow stromal cells under normoxic conditions. Embodiment 18 is the method of the method of any one of embodiments 1 to 17, wherein the method further comprises culturing the mammalian mesenchymal stem cells under normoxic conditions. Embodiment 19 is the method of the method of any one of embodiments 1 to 18, wherein the method further comprises culturing the marrow stromal cells in a container suitable for cell cultivation. Embodiment 20 is the method of embodiment 18, wherein the container is a bioreactor. Embodiment 21 is the method of the method of any one of embodiments 1 to 20, wherein the method further comprises culturing the mesenchymal stem cells in a container suitable for cell cultivation. Embodiment 22 is the method of embodiment 21, wherein the container is a bioreactor. Embodiment 23 is a method of maintaining and expanding mammalian mesenchymal stem cells in culture in an undifferentiated state, the method comprising obtaining marrow stromal cell derived 3D extracellular matrix coated microcarriers; and culturing the mammalian mesenchymal stem cells in the presence of the extracellular matrix coated microcarriers, wherein the extracellular matrix coating restrains differentiation of the mammalian mesenchymal stem cells. Embodiment 24 is the method of embodiment 23, wherein the extracellular matrix coating comprises collagen alpha-1(XII), collagen alpha-3(VI), EMILIN-1, serpin H1, thrombospondin-1, tenascin precursor (TN) (Human), transforming growth factor-beta-induced protein, and vimentin. Embodiment 25 is the method of embodiment 24, wherein the extracellular matrix coating further comprises type I collagen, type III collagen, fibronectin, decorin, biglycan, perlecan, and laminin. Embodiment 26 is the method of embodiment 25, wherein the extracellular matrix coating further comprises at least one of syndecan-1, collagen type V, or collagen type VI. Embodiment 27 is the method of any one of embodiments 23 to 26, wherein the 3D extracellular matrix is derived from murine, rabbit, cat, dog, pig, or primate marrow stromal cells. Embodiment 28 is the method of any one of embodiments 23 to 26, wherein the 3D extracellular matrix is derived from human marrow stromal cells. Embodiment 29 is the method of any one of embodiments 23 to 28, wherein the 3D extracellular matrix is derived from isolated marrow mesenchymal stem cells. Embodiment 30 is the method of any one of embodiments 23 to 29, wherein the mammalian mesenchymal stem cells are obtained from bone marrow. Embodiment 31 is the method of any one of embodiments 23 to 29, wherein the mammalian mesenchymal stem cells are obtained from umbilical cord blood. Embodiment 32 is the method of any one of embodiments 23 to 31, wherein the microcarriers are spherical in shape. Embodiment 33 is the method of any one of embodiments 23 to 32, wherein the microcarriers have a positive charge. Embodiment 34 is the method of any one of embodiments 23 to 32, wherein the microcarriers have a negative charge. Embodiment 35 is the method of embodiment 32, wherein the microcarriers comprise a cross-linked dextran matrix and have a positive charge. Embodiment 36 is the method of any one of embodiments 23 to 31, wherein the microcarriers are cylindrical in shape. Embodiment 37 is the method of embodiment 36, wherein the microcarriers are fibers. Embodiment 38 is the method of embodiment 36, wherein the fibers are hollow fibers. Embodiment 39 is the method of any one of embodiments 23 to 38, wherein the method further comprises culturing the marrow stromal cells under normoxic conditions. Embodiment 40 is the method of any one of embodiments 23 to 39, wherein the method further comprises culturing the mammalian mesenchymal stem cells under normoxic conditions. Embodiment 41 is the method of any one of embodiments 23 to 40, wherein the method further comprises culturing the marrow stromal cells in a container suitable for cell cultivation. Embodiment 42 us the method of embodiment 41, wherein the container is a bioreactor. Embodiment 43 is the method of any one of embodiments 23 to 42, wherein the method further comprises culturing the mesenchymal stem cells in a container suitable for cell cultivation. Embodiment 44 is the method of embodiment 43, wherein the container is a bioreactor. Embodiment 45 is a plurality of marrow stromal cell derived 3D extracellular matrix coated microcarriers. Embodiment 46 is the plurality of marrow stromal cell derived 3D extracellular matrix coated microcarriers of embodiment 45, further comprising mammalian mesenchymal stem cells attached to the microcarriers. Embodiment 47 is the plurality of marrow stromal cell derived 3D extracellular matrix coated microcarriers of embodiment 46, wherein the mammalian mesenchymal stem cells are attached to the 3D extracellular matrix. Embodiment 48 is the plurality of marrow stromal cell derived 3D extracellular matrix coated microcarriers of embodiment 45, wherein the microcarriers are free or are substantially free of marrow stromal cells. Embodiment 49 is the plurality of marrow stromal cell derived 3D extracellular matrix coated microcarriers of any one of embodiments 45 to 48, wherein the microcarriers are comprised in a composition. Embodiment 50 is the plurality of marrow stromal cell derived 3D extracellular matrix coated microcarriers of embodiment 49, wherein the composition is a cell culture media.

The term “mammal” or “mammalian” includes murine (e.g., rats, mice) mammals, rabbits, cats, dogs, pigs, and primates (e.g., monkey, apes, humans). In particular aspects in the context of the present invention, the mammal can be a murine mammal or a human.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of the word “a” or “an” when used in conjunction with the terms “comprising”, “having”, “including”, or “containing” (or any variations of these words) may mean “one,” but it is also consistent with the meaning of “one or more,” “at least one,” and “one or more than one.”

The compositions and methods for their use can “comprise,” “consist essentially of,” or “consist of” any of the ingredients or steps disclosed throughout the specification.

It is contemplated that any embodiment discussed in this specification can be implemented with respect to any method or composition of the invention, and vice versa.

Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: light microscopy micrographs at 10× objective of Tube B (4,000 cells/ml seeding density) and Tube F (250,000 cells/ml seeding density) at 24 hour post-cell seeding.

FIG. 2: light microscopy micrographs at 10× objective of Tube B (4,000 cells/ml seeding density) and Tube F (250,000 cells/ml seeding density) at Day 7 Pre-Induction.

FIG. 3: light microscopy micrographs at 10× objective of Tube A (2,000 cells/ml seeding density), Tube B (4,000 cells/ml seeding density), Tube E (100,000 cells/ml seeding density), and Tube F (250,000 cells/ml seeding density) at Day 14 pre-decellularization.

FIG. 4: light microscopy micrographs at 10× objective of Tube C (8,000 cells/ml seeding density), Tube D (50,000 cells/ml seeding density), Tube E (100,000 cells/ml seeding density), and Tube F (250,000 cells/ml seeding density) at Day 14 post-decellularization.

FIG. 5: SEM micrographs of untreated control beads at 100× and 2000×.

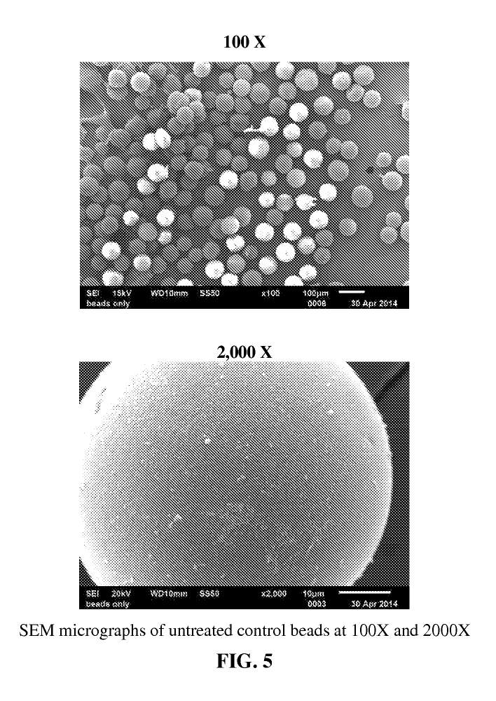

FIG. 6: SEM micrographs of Tube B (4000 cells/ml seeding density) sample at 100×, 500×, 2000×, and 5000× at Day 14 pre-decellularization.

FIG. 7: SEM micrographs of Tube B (4000 cells/ml seeding density) sample at 100×, 500×, 1000×, and 5000× at Day 14 post-decellularization.

FIG. 8: SEM micrographs of Tube D (50,000 cells/ml seeding density) sample at 1000× and 5000× at Day 14 pre-decellularization.

FIG. 9: SEM micrographs of Tube D (50,000 cells/ml seeding density) sample at 500×, 2000× and 5000× at Day 14 post-decellularization.

FIG. 10: TEM micrograph of a microcarrier bead at 40,000× at Day 14 post-decellularization.

FIG. 11: TEM micrograph of a microcarrier bead at 120,000× at Day 14 post-decellularization.

### DETAILED DESCRIPTION OF THE INVENTION

The present invention provides methods for maintaining and expanding mammalian mesenchymal stem cells (MSCs) in culture in an undifferentiated state by utilizing marrow stromal cell derived extracellular matrix (ECM) coated microcarriers as a 3D in-vitro microenvironment for the culture of the MSCs. The methods comprise first forming the ECM on the surface of the microcarriers in culture using marrow stromal cells to produce the ECM, decellularizing the ECM, and then culturing the mammalian MSCs in the presence of the marrow stromal cell derived ECM. The marrow stromal cell derived ECM promotes self-renewal of the MSCs, restrains their spontaneous differentiation toward the osteoblast lineage, and preserves their ability to differentiate into osteoblasts or adipocytes in response to BMP2 or rosiglitazone, respectively. The microcarriers coated with the marrow stromal cell derived ECM not only significantly increases the attachment surface area for adherent cells even further than with the microcarriers alone, but also allows for faster and more efficient expansion of cultured MSCs than with previous methods while maintaining the MSCs in an undifferentiated state.

### A. Marrow Stromal Cell Derived Extracellular Matrix (ECM)

The marrow stromal cell derived ECM is a three-dimensional (3D) ECM useful for maintaining the undifferentiated phenotype of MSCs and provides for the expansion of MSCs in an undifferentiated state. In the present invention, the marrow stromal cell derived ECM is coated on the surface of microcarriers.

The cells used to produce the ECM are stromal cells obtained from mammalian bone marrow. Marrow stromal cells can be obtained from various sources, such as, for example, iliac crest, femora, tibiae, spine, rib, or other medullary spaces. Marrow stromal cells can be obtained and cultured by common methods that are apparent to one of skill in the relevant art.

The marrow stromal cells contain MSCs and other cells such as fibroblasts, adipocytes, macrophages, osteoblasts, osteoclasts, endothelial stem cells, and endothelial cells. The MSCs present in bone marrow can be isolated from the other cells present in bone marrow, and the isolated MSCs can be used as the marrow stromal cells to form the marrow stromal cell derived ECM. In one embodiment, the marrow stromal cells are human. In another embodiment, the marrow stromal cells are murine, rabbit, cat, dog, pig, or primate.

The marrow stromal cell derived ECM is comprised of various proteins. The components of the marrow stromal cell derived ECM can be identified by methods known in the art and can include immunohistochemical staining and mass spectroscopy. The marrow stromal cell derived ECM, can include, but is not limited to, the following components listed in Table 1.

TABLE 1

Alpha-1-antiproteinase
Alpha-2-HS-glycoprotein
Alpha-2-HS-glycoprotein precursor
Alpha-2-macroglobulin
Alpha-actinin-1
Annexin A2
Biglycan
Caveolin-1
Collagen alpha-1(I)
Collagen alpha-1(II)
Collagen alpha-1(III)
Collagen alpha-1(VI)
Collagen alpha-1(XII)
Collagen alpha-1(XIV)
Collagen alpha-2(I)
Collagen alpha-2(V)
Collagen alpha-2(VI)
Collagen alpha-3(VI)
Collagen type I
Collagen type III
Collagen type IV
Collagen type V
Collagen type VI
Decorin
Elongation factor 1-alpha
EMILIN-1
Endoplasmin
Fibrinogen
Fibronectin
Fibulin-1
Fibulin-2
Galectin-1 - Homo sapiens (Human)
Interferon-induced GTP-binding
Lamin-A/C
Laminin
LIM domain and actin-binding protein 1
Pentraxin-related
Periostin
Periostin precursor (PN)
Perlecan
Plasminogen
Plectin
Profilin-1
Rubber elongation factor protein
Serine protease
Serpin H1
Serum albumin
Syndecan-1
Tenascin precursor (TN) (Human)
Thrombospondin-1
Transforming growth factor-beta-induced protein
Transgelin
Vimentin

The marrow stromal cell derived ECM can include any combination of components from Table 1. In particular embodiments, the combination can comprise, consist essentially of, or consist of type I collagen, type III collagen, fibronectin, decorin, biglycan, perlecan, and laminin. In another embodiment, the combination can comprise, consist essentially of, or consist of type I collagen, type III collagen, fibronectin, decorin, biglycan, perlecan, and laminin, plus can further include at least one of syndecan-1, collagen type V, or collagen type VI.

In another embodiment, the combination can comprise, consist essentially of, or consist of collagen alpha-1(XII), collagen alpha-3(VI), EMILIN-1, serpin H1, thrombospondin-1, tenascin precursor (TN) (Human), transforming growth factor-beta-induced protein, and vimentin.

In still another embodiment, the combination can comprise, consist essentially of, or consist of collagen alpha-1(XII), collagen alpha-3(VI), EMILIN-1, serpin H1, thrombospondin-1, tenascin precursor (TN) (Human), transforming growth factor-beta-induced protein, vimentin, type I collagen, type III collagen, fibronectin, decorin, biglycan, perlecan, and laminin.

In yet another embodiment, the combination can comprise, consist essentially of, or consist of collagen alpha-1(XII), collagen alpha-3(VI), EMILIN-1, serpin H1, thrombospondin-1, tenascin precursor (TN) (Human), transforming growth factor-beta-induced protein, vimentin, type I collagen, type III collagen, fibronectin, decorin, biglycan, perlecan, and laminin, plus further can include at least one of syndecan-1, collagen type V or collagen type VI.

The component profiles of the marrow stromal cell derived ECM can vary between donors of the bone marrow stromal cells, the age of the donor of the bone marrow stromal cells, and the methodology used to identify the components. As a non-limiting embodiment, the components of a “young” marrow stromal cell derived ECM from a human donor between the ages of 20-25 years old can include, but not be limited to the components from Table 2 as identified with mass spectroscopy.

TABLE 2

Alpha-1-antiproteinase
Alpha-2-HS-glycoprotein precursor
Alpha-actinin-1
Annexin A2
Biglycan
Caveolin-1
Collagen alpha-1(I)
Collagen alpha-1(VI)
Collagen alpha-1(XII)
Collagen alpha-2(I)
Collagen alpha-2(VI)
Collagen alpha-3(VI)
Elongation factor 1-alpha
EMILIN-1
Fibronectin
Fibulin-1
Galectin-1 - Homo sapiens (Human)
Lamin-A/C
LIM domain and actin-binding protein 1
Periostin precursor (PN)
Perlecan
Plasminogen
Profilin-1
Rubber elongation factor protein
Serpin H1
Serum albumin
Tenascin precursor (TN) (Human)
Thrombospondin-1
Transforming growth factor-beta-induced protein
Transgelin
Vimentin

The marrow stromal cell derived ECM can include any combination of components from Table 2. In particular embodiments, the combination can comprise, consist essentially of, or consist of collagen alpha-1(XII), collagen alpha-3(VI), EMILIN-1, serpin H1, thrombospondin-1, tenascin precursor (TN) (Human), transforming growth factor-beta-induced protein, and vimentin.

As another non-limiting embodiment, the component profile of a marrow stromal cell derived ECM from an older human donor can include, but not be limited to the components in Table 3 as identified with mass spectroscopy.

TABLE 3

Alpha-2-HS-glycoprotein
Alpha-2-macroglobulin
Biglycan
Collagen alpha-1(I)
Collagen alpha-1(II)
Collagen alpha-1(III)
Collagen alpha-1(VI)
Collagen alpha-1(XII)
Collagen alpha-1(XIV)
Collagen alpha-2(I)
Collagen alpha-2(I)
Collagen alpha-2(I)
Collagen alpha-2(I)
Collagen alpha-2(V)
Collagen alpha-2(VI)
Collagen alpha-3(VI)
EMILIN-1
Endoplasmin
Fibrinogen
Fibronectin
Fibulin-1
Fibulin-2
Interferon-induced GTP-binding
Lamin-A/C
Pentraxin-related
Periostin
Perlecan
Plasminogen
Plectin
Serine protease
Serpin H1
Serum albumin
Tenascin precursor (TN) (Human)
Thrombospondin-1
Transforming growth factor-beta-induced protein
Vimentin

The marrow stromal cell derived ECM can include any combination of components from Table 3. In particular embodiments, the combination can comprise, consist essentially of, or consist of collagen alpha-1(XII), collagen alpha-3(VI), EMILIN-1, serpin H1, thrombospondin-1, tenascin precursor (TN) (Human), transforming growth factor-beta-induced protein, and vimentin.

Another non-limiting embodiment of a marrow stromal cell derived ECM can include, but not be limited to the components from the following list as identified by immunohistochemical staining: type I collagen, type III collagen, fibronectin, decorin, biglycan, perlecan, and laminin as identified with immunohistochemical staining. Another non-limiting embodiment further comprises, consists essentially of, or consists of type I collagen, type III collagen, fibronectin, decorin, biglycan, perlecan, and laminin, plus further includes at least one of type V collagen, type VI collagen, or syndecan-1 as identified with immunohistochemical staining.

Generally, the most abundant components of a marrow stromal cell derived ECM as identified by mass spectroscopy are: collagen alpha-1(XII), collagen alpha-3(VI), EMILIN-1, serpin H1, thrombospondin-1, tenascin precursor (TN) (Human), transforming growth factor-beta-induced protein, and vimentin.

In one aspect of the invention, the marrow stromal cell derived ECM is coated on the surface of microcarriers by culturing marrow stromal cells with microcarriers in a culture medium.

### B. Mammalian Mesenchymal Stem Cells (MSCs)

Mesenchymal stem cells (MSCs) are multipotent cells that can produce daughter stem cells and can also differentiate into a variety of cell types including, but not limited to osteoblasts, stromal cells that support hematopoiesis and osteoclastogenesis, chondrocytes, myocytes, adipocytes, neuronal cells, and B-pancreatic islet cells. Mammalian MSCs mainly reside within the bone marrow, which comprises stromal cells, adipocytes, vascular elements, and sympathetic nerve cells arrayed within a complex extracellular matrix.

MSCs can be isolated from small tissue samples and expanded in-vitro under cell culture conditions. Mammalian MSCs can be obtained from various sources including, but not limited to bone marrow. Bone marrow may be obtained from various sources, such as, for example, iliac crest, femora, tibiae, spine, rib, or other medullary spaces. Mammalian MSCs can be obtained from other sources including, but are not limited to, embryonic yolk sac, placenta, umbilical cord tissues, umbilical cord blood, periosteum, trabecular bone, adipose tissue, synovium, skeletal muscle, deciduous teeth, fetal pancreas, lung, liver, amniotic fluid, and fetal and adolescent skin and blood. Methods for isolating and establishing cultures of MSCs are generally known to those of skill in the relevant art. Novel methods for isolating MSCs from umbilical cord blood are disclosed in US patent publication 2012/0142102, herein incorporated by reference in its entirety.

In one embodiment, the mammalian MSCs are human MSCs.

### C. Microcarriers

The term “microcarriers” as used herein means small support structures useful for cultivating adherent cells in culture systems. “Microcarriers” are an object or material in which at least one dimension of the object or material is equal to or less than 2500 microns and greater than 100 nm (e.g., one dimension is greater than 100 nm and less than 2500 microns in size). In a particular aspect, the microcarrier includes at least two dimensions that are equal to or less than 2500 microns and greater than 100 nm (e.g., a first dimension is greater than 100 nm and less than 2500 microns in size and a second dimension is greater than 100 nm and less than 2500 microns in size). In another aspect, the microcarrier includes three dimensions that are equal to or less than 2500 microns and greater than 100 nm (e.g., a first dimension is greater than 100 nm and less than 2500 microns in size, a second dimension is greater than 100 nm and less than 2500 microns in size, and a third dimension is greater than 100 nm and less than 2500 microns in size). The shape of the microcarrier can be of a wire, a particle, a sphere, a rod, a tetrapod, a hyperbranched structure, a cylinder (e.g., fibers, tubes, etc.) or mixtures thereof. Cylindrical shaped microcarriers can include tubular shaped microcarriers that have hollow cores (e.g., hollow fibers).

The marrow stromal cell derived ECM can be deposited on the surface of the microcarriers. Microcarriers can be made of natural or synthetic materials including, but not limited to, plastic, glass, ceramic, metal, silica, gelatin, collagen, dextran, cross-linked dextran, and cellulose. The microcarriers can be solid or porous. The microcarriers can be in any shape including, but not limited to spherical (beads) and cylindrical shapes. Cylindrical shaped microcarriers can include tubular shaped microcarriers which have a hollow core. Cylindrical shaped microcarriers can also include fibers and hollow fibers. The marrow stromal cell derived ECM can be deposited on any surface that the marrow stromal cell will attach to such as the outside surface, the inside surface, or both the outside and inside surface of tubular shaped microcarriers and hollow fiber microcarriers. The microcarriers can be positively charged, negatively charged, or have no charge. The microcarriers can be coated with a purified protein or other material to enhance cell attachment. The diameter of spherical and cylindrical shaped microcarriers generally can range from about 20 microns to about 2500 microns.

Suitable microcarriers for the present invention include positively charged spherical beads based on a cross-linked dextran matrix which is substituted with positively charged N,N-diethylaminoethyl groups. These microcarriers are available from GE Healthcare under the trade name CYTODEX 1. These microcarrier beads have a diameter of from about 150 to about 250 microns with an average diameter of about 190 microns. These microcarrier beads are biologically inert and are transparent. Other suitable microcarriers for the present invention include cylindrical hollow fibers. These hollow fibers can have a minimum inside diameter of about 10 microns.

### D. Culture Containers

Any type of container suitable for cultivation of cells can be used for the present invention. Examples include, but are not limited to cell culture flasks, T-flasks, stirred flasks, spinner flasks, fermenters, and bioreactors. Rocking bottles, shaking flasks, tubes, and other containers are also suitable containers when placed on a rocking platform or shaker to provide movement of the microcarriers. Configurations of bioreactors and fermenters include but are not limited to batch, fed batch, continuous, stirred tank, plug flow, packed bed, fluidized bed, air-lift, fluid-lift, stirred-lift, and perfusion configurations. Sizes of bioreactors and fermenters generally range from a few milliliters to 6000 liters.

### E. Culture Medium and Conditions

Various commercially available cell culture media can be used in the present invention, e.g. α-MEM culture media (Life Technologies, Thermo Fisher Scientific, Grand Island, N.Y.). The commercially available culture medium can also be modified by adding various supplemental substances to the medium, e.g. sodium bicarbonate, L-glutamine, penicillin, streptomycin, Amphotericin B and/or serum. The serum can be fetal bovine serum. Additionally, substances such as L-ascorbic acid can be added to the medium or modified medium to induce cell production of an ECM.

The culturing of the marrow stromal cells and/or the MSCs can take place under normoxic conditions, i.e. 20-21% oxygen in the atmosphere, and can further include conditions at 37° C., 5% CO2, and 90% humidity.

### F. Methods to Produce the Marrow Derived ECM on Microcarriers

The marrow derived ECM can be produced on the surface of microcarriers by the following process:
1. Obtain mammalian marrow stromal cells.
2. Add the microcarriers to a culture medium.
3. Add the marrow stromal cells to the culture medium.
4. Culture the marrow stromal cells to produce the ECM coating on the surface of the microcarriers.
5. Decellularize the ECM coated microcarriers of the marrow stromal cells.

In one aspect of the invention, the culture of the marrow stromal cells with the microcarriers takes place in a container suitable for cultivation of cells. In one embodiment, the container is a bioreactor.

In one embodiment, the culture of the marrow stromal cells with the microcarriers takes place under normoxic conditions. The ECM can be decellularized of the marrow stromal cells by using methods known in the art and can include, but are not limited to lysing the marrow stromal cells and then removing the lysed marrow stromal cells by washing. Various substances can be used to decellularize the ECM of the marrow stromal cells and include TRITON X-100 and ammonium hydroxide in PBS buffer. After the ECM is decellularized, the resulting ECM is essentially free of marrow stromal cells.

### G. Methods to Maintain and Expand Mammalian MSCs in an Undifferentiated State

Methods to maintain and expand mammalian MSCs in an undifferentiated state include obtaining mammalian MSCs and culturing them in the presence of microcarriers coated with an ECM made from marrow stromal cells.

In one aspect of the invention, the culture of the mammalian MSCs takes place in a container suitable for cultivation of cells. In one embodiment, the container is a bioreactor.

In one embodiment, the culture of the mammalian MSCs takes place under normoxic conditions.

### EXAMPLES

The following examples are included to demonstrate certain non-limiting aspects of the invention. It should be appreciated by those of skill in the art that the techniques disclosed in the examples that follow represent techniques discovered by the applicants to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Production of a Marrow Stromal Cell Derived ECM on Microcarriers

Preparation of the microcarriers for cell culture: CYTODEX 1 microcarrier beads (GE Healthcare, CAT 17-0448-01, 25 g) were chosen as the microcarriers. They are composed of a cross-linked dextran matrix and positively charged to enhance cell-surface adhesion. The beads are also biologically inert, transparent and approximately 150-250 μm in diameter. The dry beads were hydrated in PBS overnight per manufacturer's instructions (CYTODEX Instructions 18-1119-79-AD data sheet by GE Healthcare) and sterilized in an autoclave. The beads were allowed to settle and the PBS supernatant was removed by aspiration. A volume of 800 μl of hydrated beads was added to each of six 15 ml conical tubes. To allow for cell adhesion, 4 ml of a fibronectin (1 mg/ml) and PBS solution (1:60) was added to each of the conical tubes with beads and the tubes were incubated for one hour at 37° C., 5% CO2, and 90% humidity. After incubation, the supernatant was aspirated and the beads were washed two times with a PBS wash. Each wash was followed by aspiration of the supernatant. The beads were washed two additional times with α-MEM culture media (Life Technologies, Thermo Fisher Scientific, Grand Island, N.Y.). Each wash was followed by aspiration of the supernatant.

Cell seeding and culture: Seeding densities were determined by both manufacturer's directions as well as by surface area to normalize to culture methods used previously on plastic culture dishes. The manufacturer's directions suggested three densities at which the cells were seeded: A) 2,000 cells/ml, B) 4,000 cell/ml and C) 8,000 cell/ml. After initial pilot studies, three additional seeding densities were calculated based on the surface area of the beads and matched to the seeding densities used previously on plastic culture dishes. The additional three surface area based densities were calculated as D) 50,000 cells/ml, E) 100,000 cells/ml and F) 250,000 cells/ml. Each of the six conical tubes were labelled as A-E according to each cell seeding density and α-MEM culture media supplemented with sodium bicarbonate (26 mM), L-glutamine (2 mM), penicillin (100,000 I.U./L), streptomycin (100,000 μg/L), Amphotericin B (250 μg/L), and 15% fetal bovine serum (FBS) was added to each of the six conical tubes with the prepared beads. Isolated marrow mesenchymal stem cells, isolated from human bone marrow of BM donor #9602, and at passage 4, were used as the marrow stromal cells for producing the ECM on the beads. The cells were added to each of the six conical tubes at the six seeding densities stated above. The six conical tubes were then capped with T-25 cell culture caps to ensure air and humidity delivery and placed on a rocker in an incubator under normoxic conditions at 37° C., 5% CO2, and 90% humidity for 3 days. The rocking action from the rocker kept the beads from settling in the conical tubes and served to mimic their behavior in a 3-D bioreactor. On day 3, the media was changed from each conical tube by aspirating out half of the old media and adding 4 ml of fresh α-MEM culture media supplemented with sodium bicarbonate (26 mM), L-glutamine (2 mM), penicillin (100,000 I.U./L), streptomycin (100,000 μg/L), Amphotericin B (250 μg/L), and 15% fetal bovine serum (FBS). The tubes were placed back on a rocker and incubated under normoxic conditions at 37° C., 5% CO2, and 90% humidity for an additional 4 days.

Production of the ECM on the microcarrier beads: On day 7, the cells were induced to produce the ECM on the surface of the beads by aspirating out the old media from each tube and adding 4 ml of an induction media which consisted of the supplemented α-MEM culture media as described above, but which was further supplemented with 11 mg/L of L-ascorbic acid. The tubes were placed back on a rocker and incubated under normoxic conditions at 37° C., 5% CO2, and 90% humidity for an additional 3 days. On day 10, the induction media was changed in each tube by aspirating out half of the old induction media and adding 4 ml of fresh induction media. The tubes were placed back on a rocker and incubated under normoxic conditions at 37° C., 5% CO2, and 90% humidity for an additional 4 days.

Decellularizing the ECM: On day 14, the cells were removed from the ECM coated beads by adding 5 ml of a solution of 0.5% TRITON X-100 and 11 mM ammonium hydroxide in PBS buffer to each tube, and incubating for 7 minutes at room temperature. PBS was added to each tube to a final volume of 15 ml, mixed thoroughly, and then each tube was centrifuged at 400 G for 2 minutes. The supernatant was aspirated from each tube and PBS was added again to each tube to a final volume of 15 ml, mixed thoroughly, and then each tube was centrifuged again at 400 G for 2 minutes. The supernatant was aspirated from each tube and the ECM coated beads were split into two samples: post extraction bead samples for SEM and the post extraction bead samples, which were placed in new 15 ml conical tubes with 1 ml PBS plus 10 μl of 1:10 antibiotic/antimycotic for storage at 4° C. Some of the six conical tubes used for the culture exhibited cell growth on their walls. Once the beads were removed, the six conical culture tubes were filled with 10 mL PBS plus 1 ml of 1:10 antibiotic/antimycotic for storage at 4° C. for further analysis.

### Example 2

### Microscopic Analysis by Light Microscopy

Light microscopy was performed daily using a 10× objective to monitor cell growth on the beads in the six conical tubes from Example 1. Micrographs were taken of the beads directly in the conical tubes at 24 hours post-seeding, at day 7 just prior to cell induction, at day 14 just prior to decellularization and again at day 14 post decellularization. Beads were also viewed on slides using the same 10× objective by pipetting 10 μl of the beads onto a glass slide and micrographs were taken of the beads on the slides at day 7 just prior to cell induction, at day 14 just prior to decellularization and again at day 14 post decellularization.

Each conical tube was labeled according to its seeding density as previously described: A) 2,000 cells/ml, B) 4,000 cell/ml, C) 8,000 cell/ml, D) 50,000 cells/ml, E) 100,000 cells/ml and F) 250,000 cells/ml. Throughout the study, the lower 3 densities (A-C) exhibited growth of cells on the beads as observed by light microscopy. Additionally, there was little cell growth on the walls of the conical tubes until day 13 and day 14 on B. However, the higher densities (D-F) exhibited very fast cell growth from day 5 to day 12. However, over time, the beads exhibited clumping, with ECM being deposited around the beads in the clumps, and as the ECM deposits increased, the bead clumping increased. With the clumping, some beads within the clumps had cells on their surface, but other beads appeared devoid of cells. By day 7, a higher number of cells was evident on the walls of the conical tubes than on the beads in E and F, and by day 12, in D. Table 4 below is an estimation of the percent of beads covered by marrow stromal cells based on light microscopy observations and describes the observational changes in cells-on-bead growth over time. Table 5 below is an estimation of the percent of tube wall covered by marrow stromal cells based on light microscopy observations and describes the observational changes in cells-on-tube wall growth over time. Without being held to any theory, it is believed that after the ECM is laid, the cells are too confluent to continue expanding on the beads and migrate to the walls of the tube to continue expansion.

TABLE 4
(Estimation of the percent of beads covered by
marrow stromal cells based on light microscopy)

| Conical | % of Beads Covered | | | | | | | |
|---|---|---|---|---|---|---|---|---|
| Tube | Day 5 | Day 6 | Day 7 | Day 8 | Day 9 | Day 12 | Day 13 | Day 14 |
| A | <10 | <10 | <10 | <10 | 10 | 15 | 20 | 20 |
| B | <10 | <10 | <10 | 10 | 15 | 50 | 70 | 80 |
| C | 10 | 10 | 15 | 20 | 20 | 50 | 50 | 40 |
| D | 70 | 90 | <90 | <90 | <90 | <90 | 80* | 70* |
| E | 90 | <90 | <90 | <90 | <90 | 10* | 10* | <10* |
| F | <90 | <90 | <90 | 50* | 50* | 10* | <10* | <10* |

*Denotes observation of cell coverage changes from beads to walls in conical tubes.

TABLE 5
(Estimation of the percent of tube wall covered by
marrow stromal cells based on light microscopy)

| Conical | % of Tube Wall Covered | | | | | | | |
|---|---|---|---|---|---|---|---|---|
| Tube | Day 5 | Day 6 | Day 7 | Day 8 | Day 9 | Day 12 | Day 13 | Day 14 |
| A | 0 | 0 | 0 | 0 | 0 | 0 | 0 | 0 |
| B | 0 | 0 | 0 | 0 | 0 | 0 | 10 | 10 |
| C | 0 | 0 | 0 | 0 | 0 | 0 | 0 | 0 |
| D | 10 | 10 | 15 | 15 | 10 | >50 | >50 | >50 |
| E | 15 | 50 | >50 | >50 | >50 | >50 | >50 | >50 |
| F | 50 | >50 | >50 | >50 | >50 | >50 | >50 | >50 |

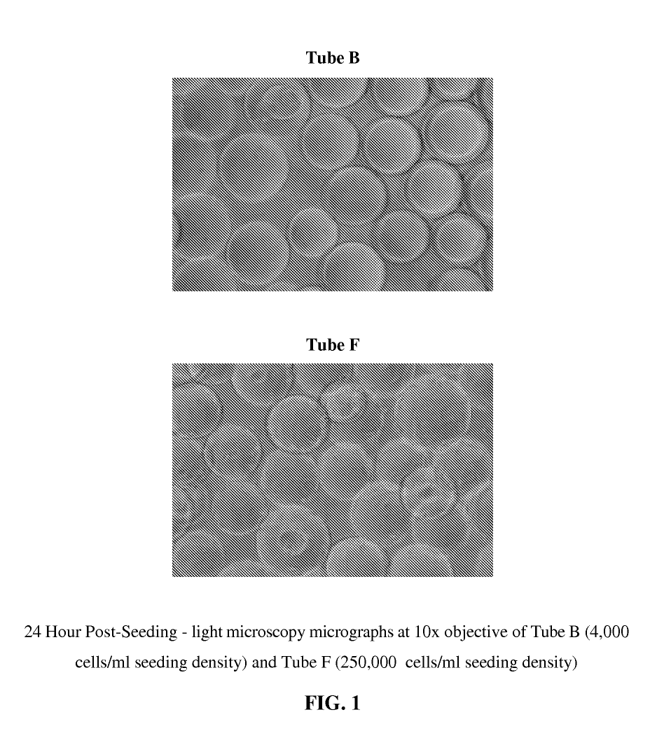

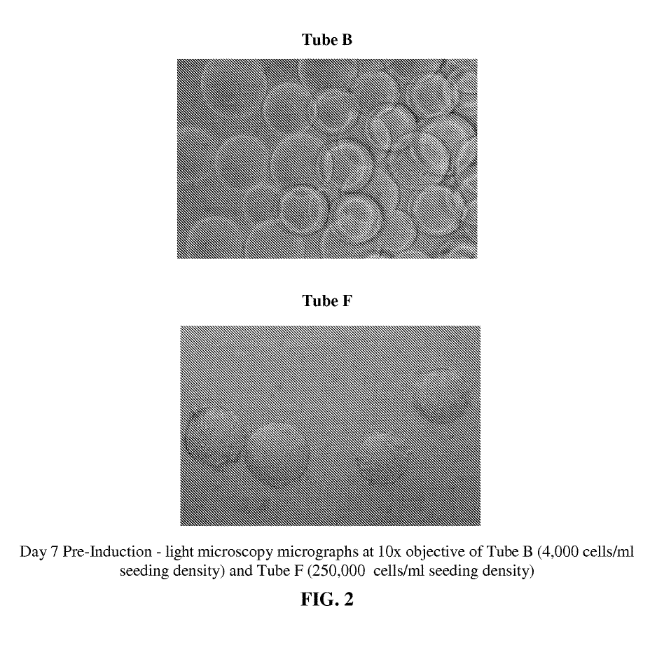

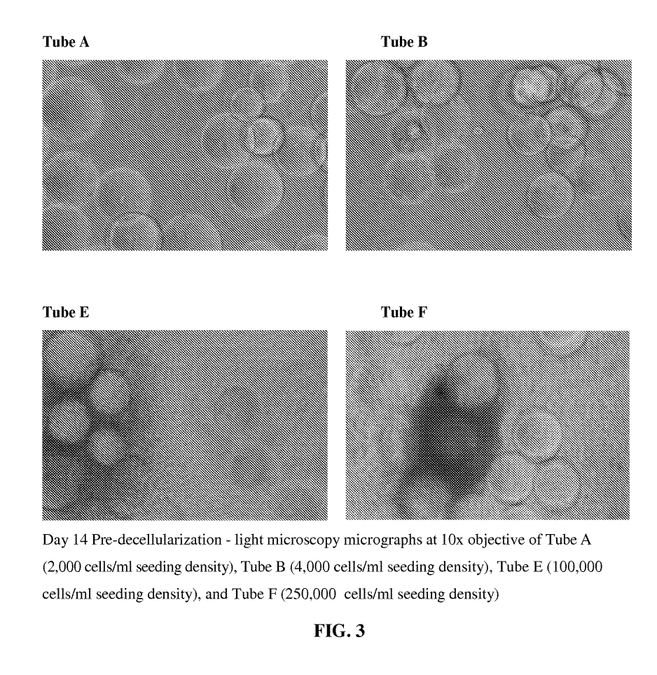

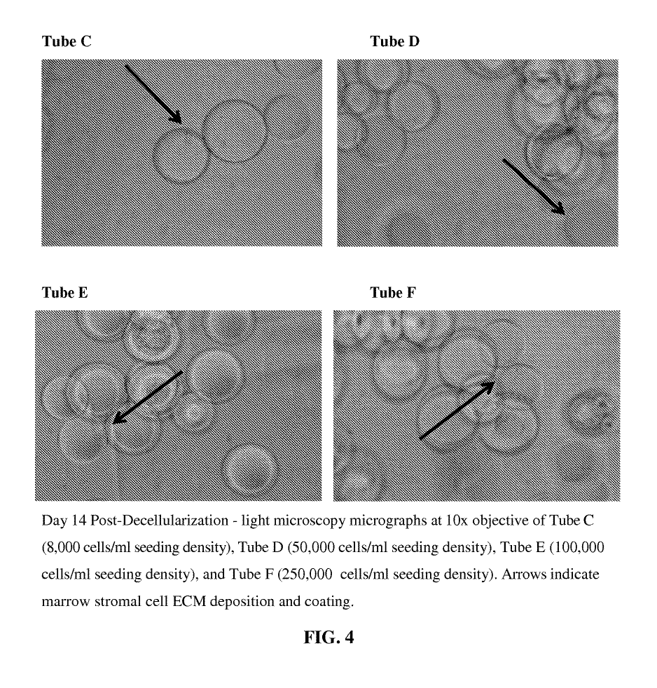

Micrographs (light microscopy at 10× objective) of the beads at various time points are shown in FIGS. 1-4. As indicated in FIG. 4, the marrow stromal cell ECM has deposited and coated the beads (see arrows on the micrographs).

### Example 3

### Microscopic Analysis by Scanning Electron Microscopy (SEM)

Samples of the beads plus supernatant were taken from each of the six conical tubes from Example 1 on day 7 just prior to cell induction, on day 14 just prior to decellularization, and on day 14 post decellularization for scanning electron microscopy (SEM). All bead samples were thoroughly mixed and 1 ml of each sample was used for SEM fixation. The beads were allowed to settle and the supernatant aspirated. The beads were then washed three times with room temperature PBS with the supernatant aspirated between each wash. After the last aspiration of PBS wash, the beads from each sample were each suspended in 1 ml of a fixation solution of phosphate buffer, formaldehyde (4%) and glutaraldehyde (1%). Samples of untreated beads were also included. Samples were labeled and stored in 4° C. refrigeration until processing and evaluation at The Pathology Electron Microscopy Facility at The University of Texas Health Science Center San Antonio.

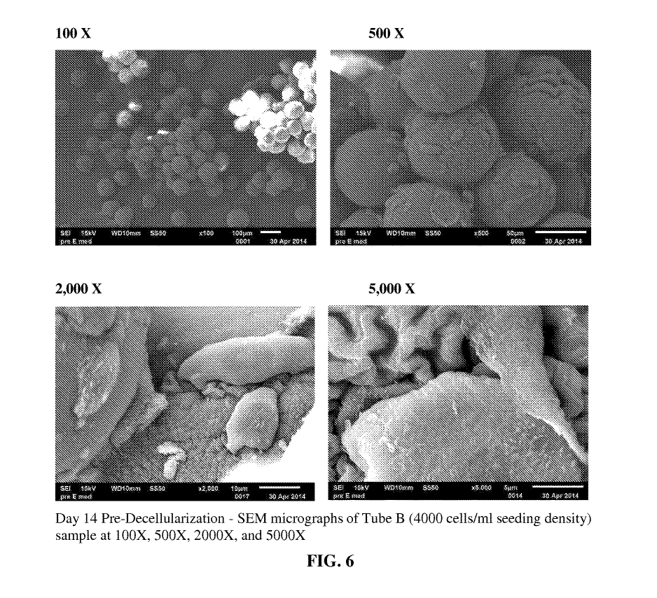

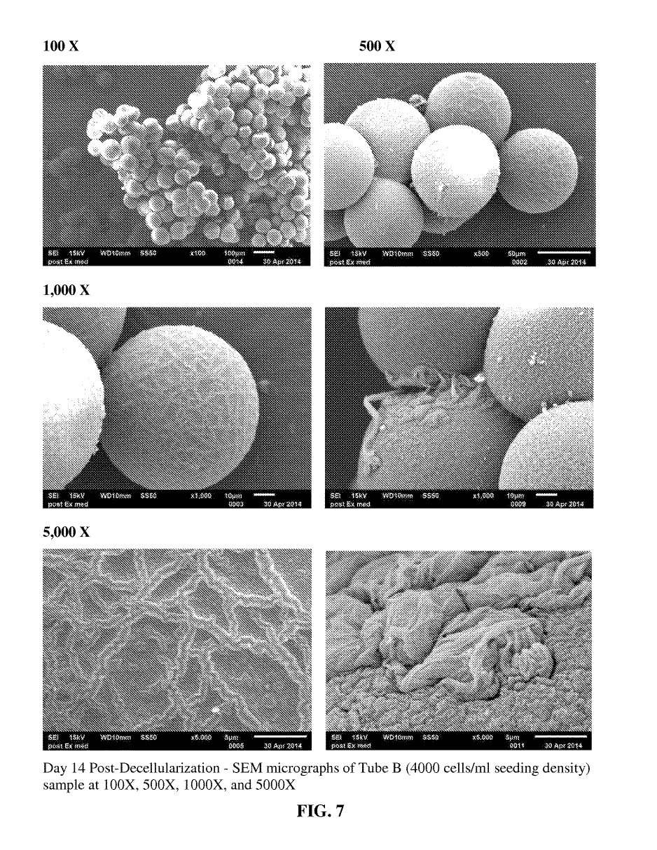

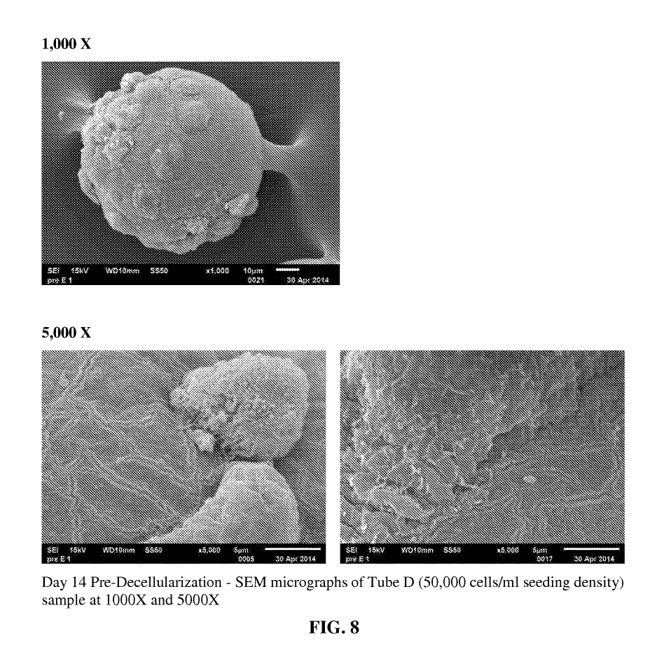

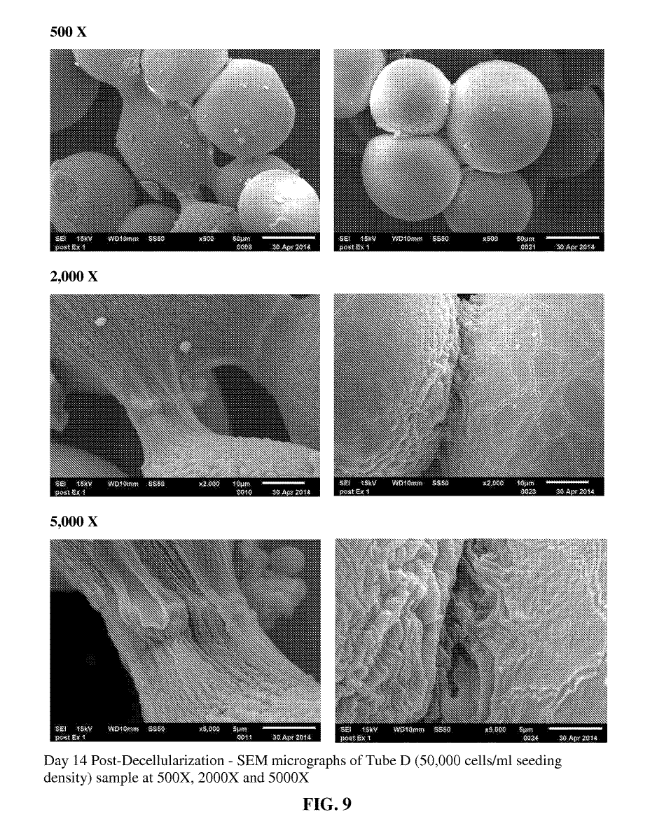

Scanning electron microscopy shows that the marrow stromal cell derived ECM was laid down on the beads, as seen in micrographs in FIGS. 6-9.

### Example 4

### Microscopic Analysis by Transmission Electron Microscopy (TEM)

Samples of Day 14 post decellularization microcarrier beads from Example 1 were analyzed by Transmission Electron Microscopy (TEM) at 40,000× and 120,000× magnification.

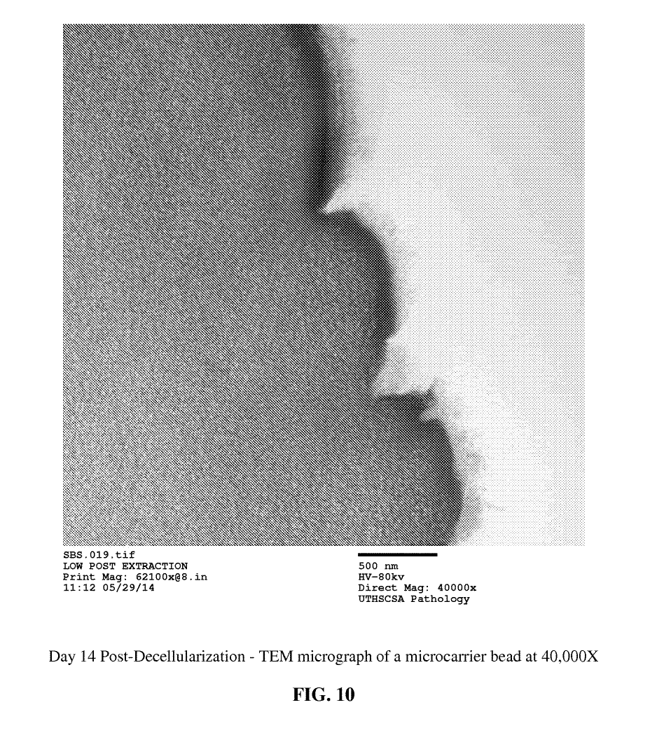

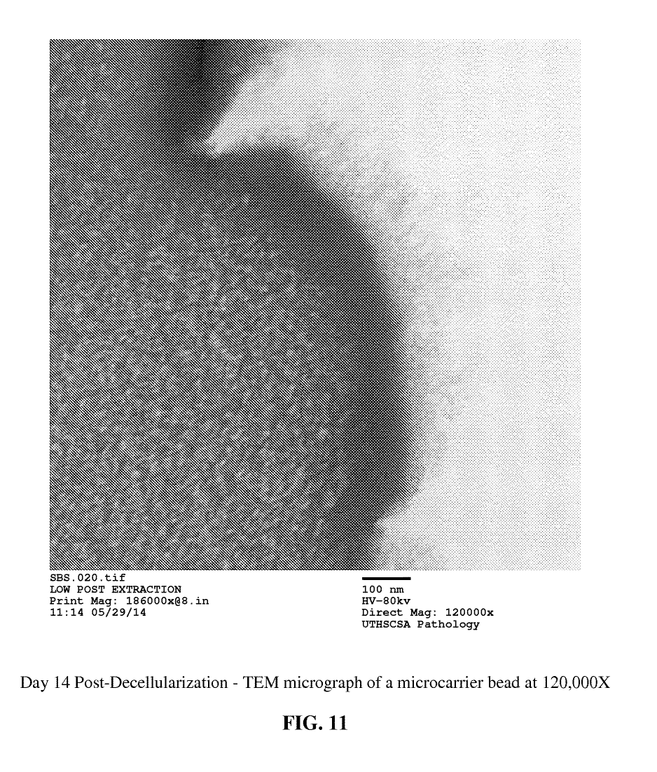

TEM shows that the marrow stromal cell derived ECM was laid down on a microcarrier bead, as seen in micrographs in FIGS. 10 and 11.

### Example 5

### Determination of Marrow Stromal Cell Derived ECM Composition Using Immunohistochemistry

Marrow stromal cell derived ECM that is coated on microcarrier beads, before or after decellularization, is fixed for about 30 minutes with 4% formaldehyde in PBS at room temperature, is washed with PBS, and is blocked with 5% normal goat serum containing 0.1% BSA in PBS for about one hour. The ECM coated microcarriers are then incubated with the relevant primary antibodies (1:10 dilution) in 2% goat serum for about two hours. Antibodies against biglycan, collagen type I, III, V, VI, fibronectin, decorin, perlecan, syndecan-1, and laminin are obtained. Non-specific isotype IgG (1:10 dilution) is used as a negative control. After washing with PBS, samples are incubated with the appropriate horseradish peroxidase-conjugated secondary antibody (1:100 dilution) for about one hour, are developed with a 3,3′-diaminobenzidine substrate-chromogen system for about five minutes, and then are counterstained with methyl green.

### Example 6

### Maintenance and Expansion of MSCs on Marrow Stromal Cell Derived ECM Coated Microcarriers

Mammalian MSCs are added to a suitable culture container containing a culture medium and the marrow stromal cell derived ECM coated microcarriers, and are incubated under normoxic conditions for a period of time. The MSCs are expanded and the undifferentiated phenotype of the MSCs is maintained throughout the culture period. MSCs from different sources may require different culture conditions.

1. A method of maintaining and expanding mammalian mesenchymal stem cells in culture in an undifferentiated state, the method comprising:
a. producing a 3D extracellular matrix coating on the surface of microcarriers comprising:
i. adding the microcarriers to a culture medium;
ii. adding mammalian bone marrow stromal cells to the culture medium;
iii. culturing the bone marrow stromal cells to produce the 3D extracellular matrix coating on the surface of the microcarriers;
iv. decellularizing the extracellular matrix coated microcarriers of the bone marrow stromal cells; and
b. culturing the mammalian mesenchymal stem cells in the presence of the extracellular matrix coated microcarriers;
wherein the extracellular matrix coating restrains differentiation of the mammalian mesenchymal stem cells.

2. The method of claim 1, wherein the extracellular matrix coating comprises collagen alpha-1 (XII), collagen alpha-3 (VI), EMILIN-1, serpin H1, thrombospondin-1, tenascin precursor (TN) (Human), transforming growth factor-beta-induced protein, and vimentin.

3. The method of claim 2, wherein the extracellular matrix coating further comprises type I collagen, type III collagen, fibronectin, decorin, biglycan, perlecan, and laminin.

4. The method of claim 3, wherein the extracellular matrix coating further comprises at least one of syndecan-1, collagen type V, or collagen type VI.

5-6. (canceled)

7. The method of claim 1, wherein the bone marrow stromal cells are isolated bone marrow mesenchymal stem cells.

8. The method of claim 1, wherein the mammalian mesenchymal stem cells are obtained from bone marrow or umbilical cord blood.

9. (canceled)

10. The method of the method of claim 1, wherein the microcarriers are spherical in shape.

11. The method of the method of claim 1, wherein the microcarriers have a positive charge.

12. (canceled)

13. The method of claim 11, wherein the microcarriers comprise are comprised of a cross-linked dextran matrix.

14. The method of the method of claim 1, wherein the microcarriers are cylindrical in shape.

15. The method of claim 14, wherein the microcarriers are hollow fibers.

16. (canceled)

17. The method of the method of claim 1, wherein the method further comprises culturing the bone marrow stromal cells under normoxic conditions.

18. The method of the method of claim 1, wherein the method further comprises culturing the mammalian mesenchymal stem cells under normoxic conditions.

19-22. (canceled)

23. A method of maintaining and expanding mammalian mesenchymal stem cells in culture in an undifferentiated state, the method comprising:
a. obtaining bone marrow stromal cell derived 3D extracellular matrix coated microcarriers; and
b. culturing the mammalian mesenchymal stem cells in the presence of the extracellular matrix coated microcarriers,
wherein the extracellular matrix coating restrains differentiation of the mammalian mesenchymal stem cells.

24. The method of claim 23, wherein the extracellular matrix coating comprises collagen alpha-1(XII), collagen alpha-3(VI), EMILIN-1, serpin H1, thrombospondin-1, tenascin precursor (TN) (Human), transforming growth factor-beta-induced protein, and vimentin.

25. The method of claim 24, wherein the extracellular matrix coating further comprises type I collagen, type III collagen, fibronectin, decorin, biglycan, perlecan, and laminin.

26. The method of claim 25, wherein the extracellular matrix coating further comprises at least one of syndecan-1, collagen type V, or collagen type VI.

27-44. (canceled)

45. A plurality of bone marrow stromal cell derived 3D extracellular matrix coated microcarriers.

46. The plurality of bone marrow stromal cell derived 3D extracellular matrix coated microcarriers of claim 45, further comprising mammalian mesenchymal stem cells attached to the plurality of extracellular matrix coated microcarriers.

47. (canceled)

48. The plurality of bone marrow stromal cell derived 3D extracellular matrix coated microcarriers of claim 45, wherein the extracellular matrix coated microcarriers are free or are substantially free of bone marrow stromal cells.

49. The plurality of bone marrow stromal cell derived 3D extracellular matrix coated microcarriers of claim 45, wherein the extracellular matrix coated microcarriers are comprised in a composition.

50. The plurality of bone marrow stromal cell derived 3D extracellular matrix coated microcarriers of claim 49, wherein the composition is a cell culture media.
